(19) United States
(12) Patent Application Publication
(10) Pub. No.: US 2005/0197553 A1
(43) Pub. Date: Sep. 8, 2005

(54) Patient management of diabetes treatment

(76) Inventor: Colleen Cooper, Minneapolis, MN (US)

Correspondence Address: SCHWEGMAN, LUNDBERG, WOESSNER & KLUTH, P.A., P.O. BOX 2938, MINNEAPOLIS, MN, 55402-0938, US

(21) Appl. No.: 10/626,942

(22) Filed: Jul. 25, 2003

Related U.S. Application Data
(60) Provisional application No. 60/399,553, filed on Jul. 30, 2002.

(51) Int. Cl. A61B 5/00; G06F 19/00; G01N 33/48; G01N 33/50

(52) U.S. Cl. 600/365; 128/921; 702/19

(57) ABSTRACT

A portable diabetes management device calculates medication dosages from glucometer readings and data such as food intake entered by the patient, according to a management plan that can be personalized by a health-care professional using a template. The device may also store and communicate past data and plan revisions.

This application claims priority to U.S. Provisional Application No. 60/399,553 filed Jul. 30, 2002.

### BACKGROUND

The present invention relates to devices and methods for assisting patients in the treatment of chronic disease, particularly diabetes mellitus.

Treating chronic diseases such as diabetes often places the patients themselves in a central role. Physicians and other professional health-care personnel cannot provide the day-to-day and even hour-by-hour measurements and dosage decisions required to maintain people with insulin-dependent diabetes functioning at an acceptable level of control. Glucose measurement, insulin formulations and delivery, and other aspects have improved over the years to the point where good control and near-normal lifestyles become more feasible. For example, some management plans employ variable or sliding-scale insulin dosages, where each short-acting insulin dose can treat a contemporaneously measured glucose level. One regimen additionally allows dosage variations for different carbohydrate intake on a meal-by-meal basis.

These improvements, however, intensify the knowledge and participation required from the patients. They must remember their own individually determined basal dosages, incremental or sliding-scale amounts for specific measured glucose ranges, and insulin equivalents for carbohydrate exchanges—including circadian variations—that can vary among individual patients. Periods of high physical exertion may decrease insulin requirements. Sick days are problematic, especially where nausea or other conditions interfere with planned meals and activities. Some people with diabetes also perform pattern adjustments on their own, when trends develop over periods of days or weeks to change the medication levels necessary for good (normoglycemic or near-normoglycemic) control. Dosage accuracy is important. The current treatment standard is geared to achieve near-normal blood-glucose levels as often as possible.

Remembering dosage factors and amounts, and repeatedly calculating and recording them, becomes burdensome for many patients. Devices such as personal blood-glucose meters (glucometers) now store measurements along with their times and dates, so that patients and physicians can review histories, or even upload a month's data into a separate desktop computer for graphing with a standard application. But conventional portable devices do not offer contemporaneous or real-time assistance in determining dosage amounts, or accommodate personal individualized treatment plans. People with diabetes are saddled with mental tasks that are inconvenient and error-prone, and that may preclude patients from taking advantage of desirable management plans merely because of their complexity.

Sufferers of diabetes still lack a convenient portable device for assisting them in carrying out personalized management plans or treatment algorithms that operate upon real-time physical measurements and data to generate dosage amounts for contemporaneous administration of medication by injection, constant infusion (insulin pump), or other routes. The term “contemporaneous” denotes times and intervals that are too short or otherwise inconvenient for patients to consult a health-care professional to determine a medication amount for a specific administration. For example, a prandial injection of regular, lispro, or other short-acting insulin to compensate for an elevated glucose or ketone level commonly follows a real-time measurement by only a few minutes. Patients may decide to alter carbohydrate intake or physical activity levels within a few minutes to hours from the insulin administration relevant to the corresponding event. Patients whom health-care professionals trust to manipulate dosages for base-level trends commonly consider a few days of measurement data to determine a contemporaneous basal injection of ultralente, glargine, or other long-acting insulin or derivative, or to adjust basal rates of an insulin pump. “Real-time” measurements refer to those that can vary significantly over time periods conformable with an interval between successive insulin administrations or boluses, such as a capillary-blood glucose test with a glucometer. (Non-realtime measurements such as glycosolated hemoglobin reflect average glucose levels over periods much longer than a few intervals between successive administrations.) A “portable” device in this context is one that patients can carry with them on a day-to-day basis without undue interference in most everyday activities—for example, approximately the size and weight of a battery-operated handheld or pocketable personal digital assistant (PDA).

### SUMMARY OF THE INVENTION

The present invention offers a portable device for assisting in the personalized treatment of diabetes in a patient, including a memory for storing an individualized reprogrammable treatment algorithm or management plan for determining dosages of a medication and for storing patient data for use by the algorithm; an instrument for measuring a real-time blood-glucose level in the patient to produce a portion of the patient data; an input device for contemporaneously receiving another portion of the patient data directly from the patient; a processor for determining a contemporaneous dosage of the medication by applying the individualized algorithm to at least some of the patient data; an output device for returning the dosage to the patient; a communications port for downloading the reprogrammable treatment algorithm into the memory; and one or more enclosures for the foregoing, in an overall portable package sufficiently small and light to be carried about by the patient.

Among many optional features, the device memory may be volatile or non-volatile, and may also store past patient data. Past data may be displayed at the patient's request, and may by uploaded to another device or computer via a communications port such as an external or built-in modem, or a port for a protocol such as RS-232 or USB. The instrument may include a glucometer for reading blood-glucose levels using conventional disposable test strips using glucose reductase or other suitable enzymes. The medication may comprise insulin, in short-acting or long-acting formulations or both. The input device may be a full or partial (e.g., numeric) keyboard or a touch screen. The processor may include or connect to an internal clock supplying current time/date information associable with measurements or other data. The output device may display characters or visual images or voice, including a representation of the current dosage calculated by the processor. An internal database may hold carbohydrate content of common foods, displaying them upon request from the patient via a pull-down menu or other means. Additional programming may also graph or tabulate glucose levels with concurrent insulin dosages and carbohydrate intake on the output device, upon request by the patient or a health-care provider.

A single integral enclosure may hold all the components of the device, including a power supply such as a battery. One of a number of physical packaging alternatives implements the memory, processor, and input/output devices as a PDA for storing the algorithm and data among other application programs, and to construct the glucometer as an external add-on employing an accessory slot in the PDA. Other chronic diseases requiring extensive patient involvement in measurement and medication administration may also benefit from variations of the invention.

The invention further extends to methods for assisting a patient in managing the treatment of diabetes or other chronic diseases, including loading a treatment algorithm or management plan personalized for a particular patient into a portable device; measuring a contemporaneous physical condition of the patient in the same device; receiving contemporaneous data directly from the patient; executing the algorithm upon a measurement of the condition and upon the received patient data so as to determine a dosage of a medication for contemporaneous administration to the patient; and outputting the dosage to the patient. Optional features may include prompting patients for data, requesting confirmation of calculated dosages, and checking calculated dosages against boundary conditions for safety.

A health-care professional may personalize the algorithm for the particular patient from a template, usually located in another computer or in the professional's office. The template may name certain variables or slots whose values are specified by the professional for the individual patient. This personalized or individualized algorithm is then downloaded to the patient's device, either locally in the office or via a network such as a public telephone or the Internet, via a communications port on the device. Patient data for personalizing the algorithm may be developed in personal appointments with the professional, from diagnostic tests, and/or from stored patient data uploaded or otherwise transmitted from the device. The professional may review and revise the algorithm in the same manner, and may request that the patient upload stored data periodically. Alternatively, patients themselves may adjust their algorithms for short-acting insulin when needed, after guidance from a diabetes educator. Some patients may be able to personalize their original algorithms or plans by themselves. In this case, the patient's device may contain a template and programming to display it, receive variable values on the device's keyboard or other input modality, and enter the values into the plan.

### BRIEF DESCRIPTION OF THE DRAWING

FIG. 1 is a block diagram of an example of a diabetes management device according to embodiments of the invention.

FIG. 2 is a flowchart showing examples of methods according to embodiments of the invention.

### DESCRIPTION OF EMBODIMENTS

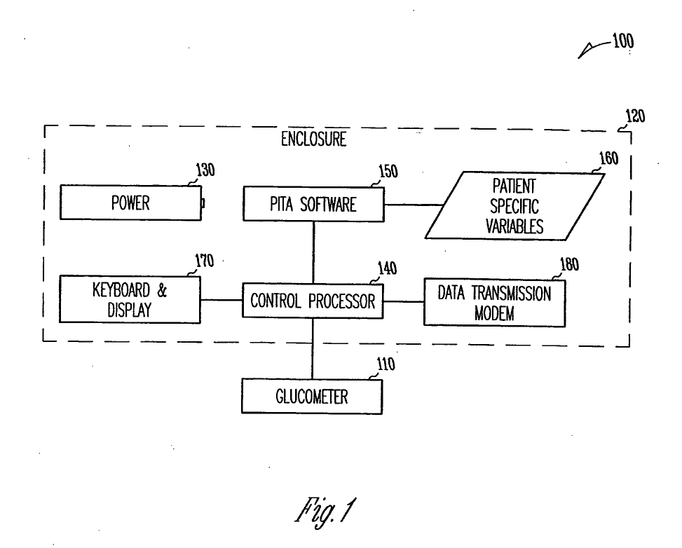

FIG. 1 illustrates a portable device 100 for realizing one form of the invention using a personalized management plan employing an insulin treatment algorithm (PITA). One type of PITA is called functional insulin treatment (FIT); this description may employ both terms.

Device 100 assists in treating and managing diabetes. The device is a handheld electronic device about the size of a personal digital assistant or a cellular telephone. The device integrates the following functions.

Device 100 measures, displays and stores the level of the patient's blood glucose. Components that perform this function, i.e., glucose meters 110, are readily available in the marketplace. The device could incorporate existing glucometer technology. Currently available glucose meters offer a function which records and graphs the glucose data generated. The glucometer could be an integral part of the device, or it could be a separate product that plugs into the device, for example in a standard format of an accessory card using an accessory slot of a personal digital assistant. (Alternatively, all elements of the device could be housed in a single hand-held enclosure such as 120, along with a battery 130 or other power source.)

A small computer 140 includes memory loaded with software 150 that integrates a PITA with the patient's glucose measurement, including patient-specific variable values 160. The PITA is pre-established by the patient's health care provider. The patient's PITA is used to determine the amount of insulin the patient should inject at meals or for correction of glucose elevations or depressions outside the target range. Each device is programmed with the patient's specific PITA, which may be adjusted as necessary for optimal blood glucose control. After a blood glucose test is completed, the device displays on a combined input/output device 170 a series of questions and prompts that guide the computation of the insulin dose to be injected at that time. After the dose has been computed, computer 140 evaluates the dose against the patient's PITA and other pertinent data. If the dose appears to be in the correct range, the device displays that information and asks the patient to confirm the dose. If the dose appears to be incorrect, the device will advise the patient of the possible error.

Device 100 has the capacity to transmit the patient's blood glucose levels via a data transmission line such as modem 180 to the patient's health care provider for review and follow-up. If the device is packaged as a multi-purpose device such as a personal digital assistant, modem 180 or a similar device may upload instructions and data for software 150 from a communications medium or a storage medium. Technology for transmitting the data electronically is readily available for this application.

One feature of the device is the integration of the patient's glucose measurements with the PITA, allowing easy dosage adjustments for food intake and for the patient's existing glucose level. This integration of functions facilitates two aspects of current diabetes treatment and management. The first is an insulin regimen more precisely tailored to the patient's specific needs, allowing tight glucose control and an ability to accommodate variations in the patient's diet and level of activity. The second is periodic remote monitoring of the patient's control of glucose levels without needing frequent clinic visits. In 1993, the Diabetes Complication and Control Trial was published. This study definitely proved that tight glucose control delays or slows the development of diabetic complications; continuation of this study has recently confirmed these findings. Unfortunately, the research protocol was sufficiently intense in terms of medical staff time and overall expense that it has been very difficult for that level of glucose control to be replicated in the standard clinic setting. Device 100 provides a systematic protocol for achieving tighter glucose control and regular provider interaction without an excessive demand for staff time. Although some staff time might be displaced by in-depth training of the patient in diabetes management, the device facilitates this training.

Intensive insulin therapy has been the ideal standard for diabetes care over the past 25 years. Currently, patients using the PITA approach to manage their disease must rely on written forms for dosage calculation and record keeping. Electronically integrating the patient's PITA into this device greatly enhances the precision, accuracy, reliability and ease of use of the PITA approach. The device also assists in the preparation of patients for greater self-management of their diabetes.

Vesting greater control over the treatment of diabetes in the patient does not eliminate the need for periodic review and, if necessary, intervention by physicians and diabetes educators. The capacity of device 100 to transmit data to the clinic of the patient's health provider, however, contributes to efficient and therapeutically sound management of diabetes. Competent health professionals at the receiving end of the data can monitor the extent to which the patient is competently employing the PITA and, if necessary, suggest changes to the PITA. The availability of this technology will ultimately improve patient-provider communication and interaction without adding a significant added cost or time burden.

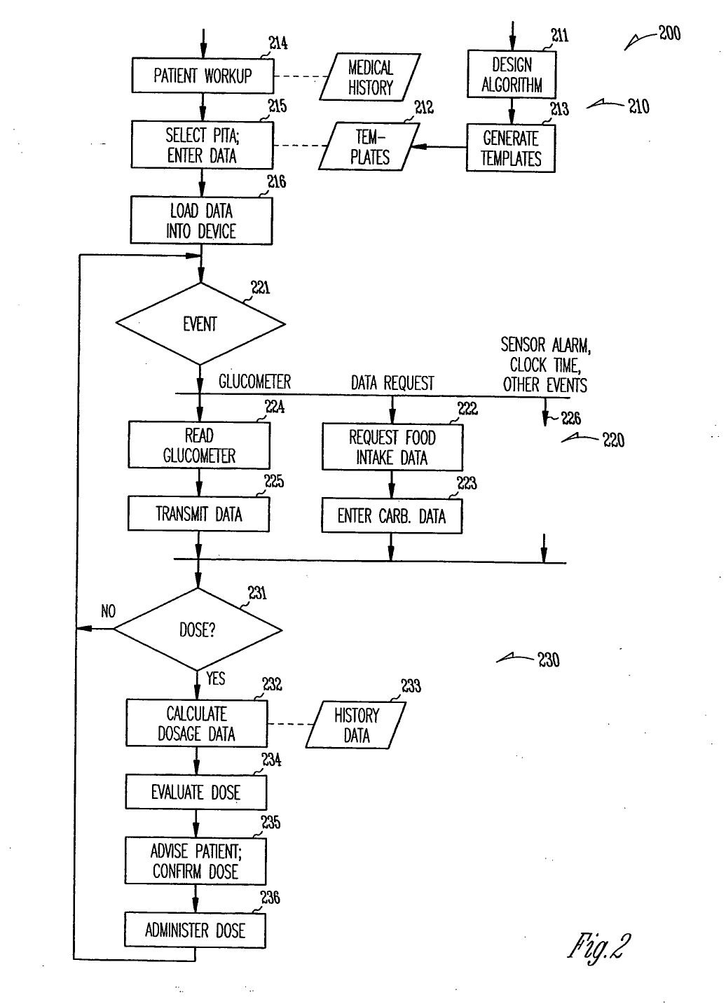

FIG. 2 shows a flowchart 200 illustrating the operation of an exemplary method according to embodiments of the invention. Some of the illustrated operations may be omitted, further operations may be included, and the operations may be performed in temporal orders different from those shown. Operations 210 may be performed by a health-care professional and/or by the manufacturer or distributor of a dedicated device 100 or application software 150, FIG. 1. The system maker designs 211 the basic personal insulin treatment algorithm (PITA), specifying which factors are included and coding the necessary calculations and data input/output routines. The system maker may also generate 212 one or more templates 213 from the design, to provide an interactive program for a health-care professional to enter data associated with particular patients.

The patient's medical history data may arise from interviews with the health-care professional, from laboratory tests, and/or from data uploaded from a device such as that described above. Workup 214 and algorithm (PITA) selection 215 blocks may occur at the professional's office or similar location. Data loading 216 may occur in the office or over a communications facility such as the Internet.

The remaining operations may be carried out by a patient's device such as 100, at any convenient location, including where the medication is to be administered. Dose administration is normally performed by the patient from an evaluated-dose display, although the device could be integrated with an insulin pump or other modality for direct infusion. Data-request blocks 220 allow the patient or the device itself to choose at 221. to perform certain operations, such as entering variations in food intake for a meal, at 222. Carbohydrate amounts entered at 223 can then be used to alter subsequent dosage calculation. Other patient history could be entered in the same manner, if desired. For example, separate urine ketone test results could be entered here. Block 224 reads a glucometer. Block 225 may transmit this reading and or others. Other operations 226 may include sounding alarms, signaling clock time, and other events.

Operations 230 determine a contemporaneous dosage when requested at 231. Block 232 employs the personalized PITA or management plan to calculate a dose of a medication such as an insulin bolus from history data 233. The history data block 233 stores patient history data, such as current and past blood-glucose readings, recorded carbohydrate intakes, and previous insulin dosages, for calculating dosage data. Patient history data could also be downloaded into the health-care professional's medical data for usages such as selecting or modifying the PITA parameters. Block 234 evaluates the calculated dosage according to limit values, etc., for safety purposes. Block 235 displays or otherwise advises the patient of the dosage amount and requests confirmation. For most present administration modalities, the patient manually administers the medication. However, block 236 may administer the medication automatically upon confirmation through an insulin pump or other means.

Those skilled in the art understand how to design a PITA or management plan for use at block 232. The book FUNCTIONAL INSULIN TREATMENT, by Kinga Howorka, M.D. (Springer Verlag, 2d English Ed., 1996), ISBN 3-540-60352-2 and 0-387-60352-2, incorporated herein by reference, illustrates representative factors and algorithms which the invention may employ in assisting patient treatment for insulin-dependent diabetes. FIG. 4.1 on page 55 of this book illustrates one form of personal insulin treatment algorithm (PITA) and a paper form that a physician or other health-care professional might employ to individualize or personalize the PITA for a particular patient in implementing a functional insulin treatment (FIT) regime. An electronic template according to the invention could employ an interactive template having a layout similar to this form for entry of individualized patient data. Such a template could be custom-coded for this application, or might be implemented with a conventional spreadsheet application program.

The foregoing description and drawings illustrate specific embodiments of the invention sufficiently to enable those skilled in the art to practice it. Other embodiments may incorporate structural, logical, electrical, process, and other changes. Examples merely typify possible variations. Individual components and functions are optional unless explicitly required, and the sequence of operations may vary. Portions and features of some embodiments may be included in or substituted for those of others. The scope of the invention encompasses the full ambit of the following claims and all available equivalents.

1. A device for assisting in the personalized treatment of diabetes by a patient, the device comprising:
a memory for storing an individualized reprogrammable management plan for determining dosages of a medication and for storing individualized real-time patient data for use by the plan;
a connection for receiving real-time blood-glucose levels in the patient from a glucometer, to produce a portion of the patient data;
an input device for receiving another portion of the patient data directly from the patient, the other portion including carbohydrate intake;
a processor for determining a contemporaneous dosage of the medication by applying the individualized plan to at least some of the data in the memory;
an output device for returning the dosage;
a communications port for downloading the reprogrammable treatment plan into the memory;
one or more enclosures for the foregoing, in an overall portable package sufficiently small and light to be carried about by the patient.

2. The device of claim 1 where the memory further stores past patient data.

3. The device of claim 2 where the past patient data includes past blood-glucose levels.

4. The device of claim 3 where the past patient data further includes past carbohydrate intake and past dosage amounts.

5. The device of claim 4 further comprising programming for presenting patient glucose values with concurrent values of medication dosage and carbohydrate intake.

6. The device of claim 1 further including a real-time clock for producing time data associable with at least some of the patient data.

7. The device of claim 1 further comprising a modem for communicating data including at least some of either or both the patient data and the management plan.

8. The device of claim 1 where the portable package has only a single integral enclosure.

9. The device of claim 8 where the single enclosure further includes the glucometer.

10. The device of claim 8 where the single enclosure further encloses a power supply.

11. The device of claim 1 where the portable package includes multiple enclosures, the glucometer residing in a separate one of the enclosures.

12. The device of claim 11 further including an accessory slot in another one of the enclosures, and where the glucometer communicates with the processor via the accessory slot.

13. The device of claim 12 where the memory, input device, processor, output device, and accessory slot are located in a personal digital assistant.

14. The device of claim 1 further comprising a database of carbohydrate values for different foods, the database being accessible via the output device.

15. A machine-implemented method for assisting a patient in managing the treatment of diabetes, comprising:
loading a treatment management plan personalized for a particular patient into a device small enough to be carried about by the patient;
measuring a contemporaneous blood-glucose level of the patient;
receiving contemporaneous data directly from the patient, at least some of the input data concerning carbohydrate intake;
executing the plan upon a measurement of the condition and upon the received patient data so as to determine an insulin dosage for contemporaneous administration to the patient;
outputting the dosage.

16. The method of claim 15 where the contemporaneous blood-glucose level is received directly from a glucometer.

17. The method of claim 15 further comprising storing past values of at least some of the contemporaneous data.

18. The method of claim 15 further comprising recalling at least some of the stored contemporaneous data.

19. The method of claim 15 further comprising receiving history data from the patient.

20. The method of claim 19 where the history data includes ketone test results.

21. The method of claim 15 where the dosage is displayed to the patient for administration.

22. The method of claim 15 where the dosage is output directly to a device capable of administering the medication.

23. A machine-readable medium bearing instructions and data for carrying out the method of claim 15 on a digital data processor.

24. A machine-implemented method for assisting a patient in managing the treatment of diabetes, comprising:
storing a template of a management plan for administration of a medication by the patient;
entering values of variables in the management plan so as to personalize the plan for the individual patient;
downloading the personalized management plan to a portable device capable of executing the plan in conjunction with other data entered by the patient on a real-time basis in order to determine dosages of the medication on a real-time basis after the download.

25. The method of claim 24 where at least some of the values of the variables are determined from non-realtime diagnostic tests of the patient.

26. The method of claim 25 further comprising receiving stored patient data from the portable device, and where the management plan is personalized at least partially from the received data.

27. The method of claim 25 further comprising, after downloading the management plan:
receiving stored patient data from the portable device;
revising the management plan in response to the uploaded data;
downloading the revised plan to the portable device.

28. The method of claim 25 further comprising receiving uploaded patient data from the portable device over a network.

29. The method of claim 24 further comprising displaying at least some of the stored patient data directly to the patient, and receiving revisions to the management plan directly from the patient.

30. The method of claim 24 further comprising generating the stored template according to an algorithm for determining dosage of the medication.
